(19) United States
(12) Patent Application Publication
(10) Pub. No.: US 2014/0288366 A1
(43) Pub. Date: Sep. 25, 2014

(54) ENDOSCOPIC SYSTEM

(71) Applicant: CANON KABUSHIKI KAISHA, Tokyo (JP)

(72) Inventor: Minoru Ohkoba, Sagamihara-shi (JP)

(73) Assignee: CANON KABUSHIKI KAISHA, Tokyo (JP)

(21) Appl. No.: 14/193,626

(22) Filed: Feb. 28, 2014

(30) Foreign Application Priority Data
Mar. 19, 2013 (JP) 2013-056034

(51) Int. Cl. A61B 1/06; A61B 1/00

(52) U.S. Cl. CPC A61B 1/06; A61B 1/00131
USPC 600/104

(57) ABSTRACT

When light reflected off a treatment tool and light reflected off an organ during in vivo observation are equal in brightness and wavelength, both the treatment tool and organ appear clear with an unwanted object acting as a disturbing element. Also, there is a problem in that a desired object cannot be observed conversely in amniotic fluid due to high transparency when fiber is used alone. A treatment tool is made of transparent material, provided with illuminating light for treatment tool observation differing at least partially in wavelength from illuminating light for in vivo observation, and configured to allow intensity to be adjusted independently. Visibility of the treatment tool in a wavelength range of the illuminating light for treatment tool observation is configured to be higher than visibility of the treatment tool in a wavelength range of the illuminating light for in vivo observation.

### BACKGROUND OF THE INVENTION

1. Field of the Invention

The present invention relates to an endoscopic system which controls visibility of a treatment tool in an endoscopic image, the treatment tool being used together with an endoscope.

2. Description of the Related Art

It is known that endoscopic procedures and endoscopic surgeries use treatment tools such as suture forceps and hemostatic laser fiber in conjunction with an endoscope. During procedures using such a treatment tool, it is sometimes difficult to observe an affected area, being blocked from view by the treatment tool. On the other hand, once the treatment tool is moved out of the field of view, it is difficult to return it to its original position. Therefore, a treatment tool that does not obstruct in vivo observation is desired.

An endoscope provided with a treatment tool insertion hole in the plane in which an objective lens is placed is known, where the treatment tool insertion hole is used to insert a treatment tool such as a laser fiber. In a single-port surgery, vascular hemostasis and the like are performed by passing an optical fiber through the treatment tool insertion hole and emitting laser from a distal end. The endoscope has been reduced in diameter to reduce invasiveness to a body, and there is a small placement distance between the objective lens and a treatment tool such as a laser fiber. Consequently, a large share of a monitor screen is occupied by the treatment tool, further obstructing the observation of the affected area.

Japanese Patent Application Laid-Open No. 2008-136671 discloses a stereoscopic endoscope provided with plural objective lenses. With Japanese Patent Application Laid-Open No. 2008-136671, when procedures are carried out by passing a treatment tool through a treatment tool insertion hole of the endoscope as described above, there is a problem in that not only a large share of the screen is occupied by the treatment tool, but also there is a large parallax between the left and right eyes with respect to the treatment tool coming out of the treatment tool insertion hole, resulting in a stronger sense of interference (FIG. 6). To solve this problem, Japanese Patent Application Laid-Open No. 2008-136671 uses a transparent material. Also, in view of the fact that if the material is completely transparent, conversely, a distal end of the laser fiber cannot be identified visually and an organ could be damaged unexpectedly, posing a danger, visibility is increased partially by coating only a distal end portion of the treatment tool with a non-translucent coating.

With the technique described in Japanese Patent Application Laid-Open No. 2008-136671, translucency varies with the position of the fiber which is a treatment tool. If a non-translucent material is used only for the distal end, when procedures are carried out with a short portion of the fiber thrust out from a distal end of the endoscope, a sense of interference is produced by the non-translucent material. That is, if a portion of translucent coating and a portion of non-translucent coating are location-dependent, a sense of interference might be produced depending on maneuvers. Thus, desirably the visibility of the treatment tool can be changed as needed and the visibility can be set for the entire treatment tool without producing a sense of interference.

### SUMMARY OF THE INVENTION

The present invention provides an endoscopic system comprising: an emission unit; and a treatment tool, wherein the emission unit emits light in a first wavelength region and light in a second wavelength region, and visibility of the treatment tool under the light in the first wavelength region is higher than visibility of the treatment tool under the light in the second wavelength region.

Further features of the present invention will become apparent from the following description of exemplary embodiments with reference to the attached drawings.

### BRIEF DESCRIPTION OF THE DRAWINGS

FIG. 1A illustrates an endoscope distal end portion and FIG. 1B illustrates a video image captured by the endoscope, the video image also containing a treatment tool.

FIG. 2A illustrates a stereoscopic endoscope distal end portion and FIG. 2B illustrates a video image captured by the endoscope, the video image also containing a treatment tool.

FIG. 3 illustrates an endoscopic system.

FIG. 4 illustrates emission spectra of illuminating lights.

FIG. 5 illustrates regular reflectances of treatment tool surfaces in Example 1 and Comparative Example 1.

FIG. 6 illustrates regular reflectances of treatment tool surfaces in Example 2 and Comparative Example 2.

### DESCRIPTION OF THE EMBODIMENTS

Preferred embodiments of the present invention will now be described in detail in accordance with the accompanying drawings.

The present invention provides an endoscopic system comprising: an emission unit; and a treatment tool, wherein the emission unit emits light in a first wavelength region and light in a second wavelength region, and visibility of the treatment tool under the light in the first wavelength region is higher than visibility of the treatment tool under the light in the second wavelength region.

The treatment tool refers to tools in general used for endoscopic procedures, including, for example, knives, forceps, fibers, extension tools, and all other accessories.

Also, regarding a surface of the treatment tool according to the present invention, a surface of a treatment tool body can be coated with a coating layer for protection and visibility improvement. The coating layer can be made of a material different in refractive index from the treatment tool body.

It is advantageous for the treatment tool to have low visibility during emission of light for in vivo observation. Thus, advisably the interior of the treatment tool body is made of translucent material, and inorganic glass made principally of SiO₂ can be used suitably, for example. A material which does not have translucency and has high internal diffuse reflectance and a material which has a special color cannot be reduced in visibility because such materials cannot be turned into a same color as a surrounding body color by adjusting regular reflection.

Although the coating layer is not particularly limited, with any material which indicates a reflectance corresponding to a wavelength as a physical property value, the visibility of the treatment tool can be changed depending on the wavelength of illumination. Possible methods for coating the treatment tool with a coating material include a sputtering process as well as a dip process in a sol-gel material followed by burning, and the dip process is used suitably. Examples of advisable material for coating layers include an ITO (indium tin oxide) layer.

The above configuration allows the treatment tool to have low visibility under light in a wavelength region (second wavelength region) for in vivo observation while having high visibility under light in a wavelength region (first wavelength region) for treatment tool observation.

Advisably the second wavelength region in the present invention is from 450 nm to 700 nm (both inclusive). There is no particular limit to the first wavelength region as long as the first wavelength does not overlap the second wavelength region.

Incidentally, the visibility of the treatment tool is the ease with which the treatment tool can be seen visually in an endoscopic system. Specifically, the visibility of the treatment tool can be determined based on the reflectance of the surface of the treatment tool body (high reflectance corresponds to high visibility) in the wavelength region of emitted light, a numeric value measured when the treatment tool is actually observed through the endoscopic system under illumination with light in the wavelength region, or intensity (e.g., in terms of the number of gradations) indicated on a monitor. A higher visibility means a higher numeric value in at least any one of the reflectance of the surface of the treatment tool body in the wavelength region of emitted light, the numeric value measured when the treatment tool is observed through the endoscopic system under illumination with the light in the wavelength region, the intensity indicated on the monitor, and numeric values equivalent to these.

Note that the treatment tool may contain a phosphor which is excited to emit light at a wavelength of 450 nm or below. In that case, the phosphor may be contained in either the treatment tool body or coating layer. Examples of the phosphor include Zn₂SiO₄.

The endoscopic system according to the present invention may emit the light in the first wavelength region and the light in the second wavelength region from a single emission unit. In that case, if a single light source is used, the wavelength can be converted using a filter. Alternatively, the endoscopic system may include a first emission unit adapted to emit the light in the first wavelength region and a second emission unit adapted to emit the light in the second wavelength region. Also, the emission unit can have a light source and an optical fiber as well as a filter adapted to selectively pass light of a specific wavelength. Besides, the emission unit may have a light-emitting element. In either case, the intensity of one or both of the light in the first wavelength region and the light in the second wavelength region may be made variable.

The endoscopic system according to the present invention may have a treatment tool insertion hole used to insert the treatment tool. Furthermore, the endoscopic system according to the present invention may have plural imaging units as well as a circuit adapted to generate a three-dimensional image based on information obtained by the plural imaging units.

Also, the present invention provides an imaging method using an endoscope equipped with an emission unit, an imaging unit, and a treatment tool, wherein the emission unit operates in a first mode in which light in a first wavelength region is emitted and a second mode in which light in a second wavelength region is emitted, the imaging method comprising a first observation step of observing the treatment tool using illumination in the first mode, and a second observation step of observing the object under observation using illumination in the second mode.

There is no particular limit to an environment in which the endoscopic system according to the present invention is used, but as a desirable example, it is assumed that the endoscopic system is used in a liquid such as amniotic fluid.

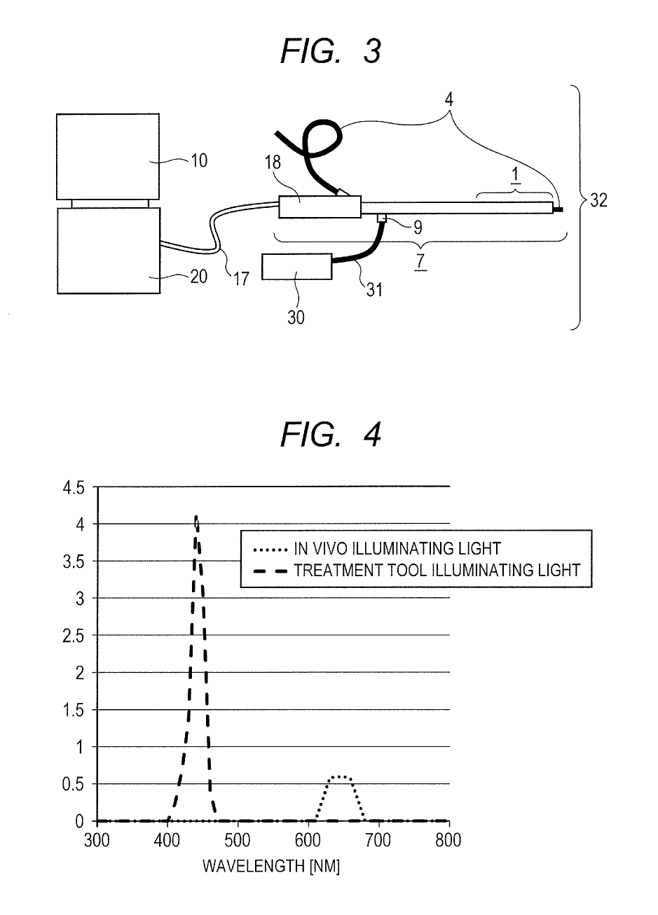

An embodiment of the present invention will be described below with reference to FIG. 3.

An example of an endoscopic system according to the present embodiment is illustrated in a diagram of FIG. 3. The endoscopic system 32 includes an endoscope 7, a display 10, a video processor 20, a signal cable 17 and a light source 30. Light from the light source 30 is guided from the light source to the endoscope distal end portion by an optical fiber 31. A combination of the light source and optical fiber 31 in FIG. 3 corresponds to the emission unit. However, the emission unit may include other components such as a filter. Also, rather than a combination of the light source 30 and optical fiber 31, an LED may be used as the emission unit. The endoscope 7 includes a control unit 18 gripped by an operator and an endoscope distal end portion 1 inserted into the body of a person to be observed with a treatment tool 4 attached thereto. An endoscope distal end portion, which contains an objective lens as well as a CCD or CMOS sensor serving as an image receiving element, is configured to be able to capture images of an object to be observed by the endoscope distal end portion 1. Alternatively, the endoscope is configured to transmit an image in the state of light from the endoscope distal end portion 1 to the control unit 18 via a fiber or relay lens, with an image receiving element installed in the endoscope control unit. The endoscope illustrated in FIG. 3 is a type in which the treatment tool insertion hole is provided in the endoscope and a treatment tool is pushed out from the distal end, but the present invention is not limited to this. However, with the endoscope of the type described above, the endoscopic system may be configured such that the treatment tool will be inserted into the body without passing through the endoscope, which does not have a treatment tool insertion hole, and will be shown in the field of view of the endoscope. The present invention has a larger effect on the endoscopic system of the type in which the treatment tool is pushed out from the endoscope distal end portion because the treatment tool occupies a larger area of the field of view in this type of endoscopic system. Also, although the endoscope illustrated in FIG. 3 is a rigid endoscope, according to the present invention, the endoscope may be either a rigid endoscope or a flexible endoscope. Also, the endoscope may be a stereoscopic endoscope provided with plural objective lenses and adapted to gain a sense of distance using an amount of parallax. To observe the interior of the body with the endoscope, the endoscope distal end portion 1 is inserted into the body.

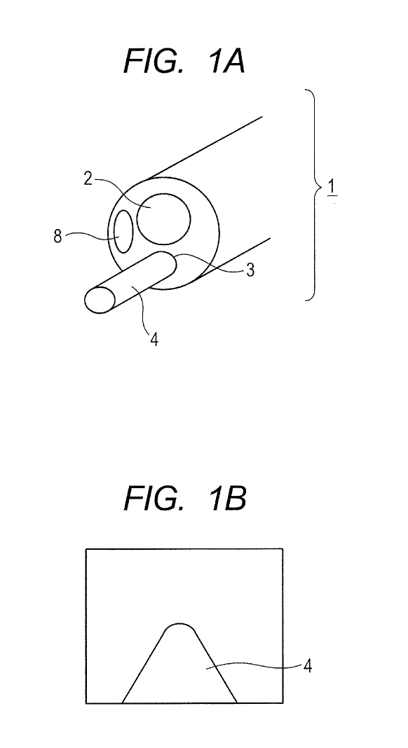

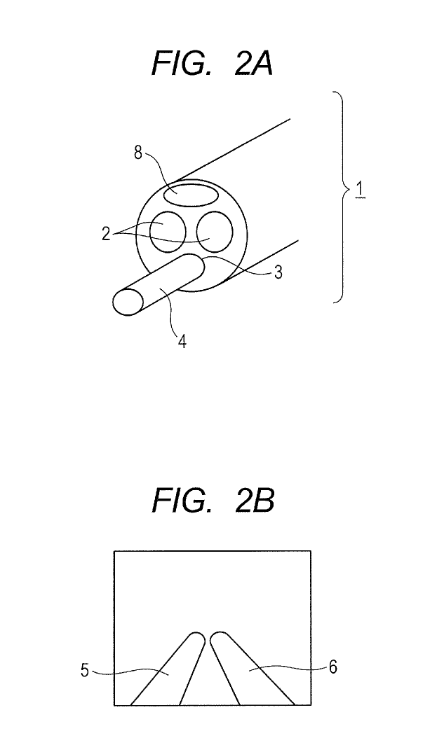

The endoscope distal end portion will be described in more detail with reference to FIGS. 1A to 3. FIGS. 1A and 1B illustrate an example of a monocular endoscope while FIGS. 2A and 2B illustrate an example of a compound-eye endoscope. FIG. 1A illustrates an endoscope distal end portion and FIG. 1B illustrates a video image captured by the endoscope, the video image also containing a treatment tool. FIG. 2A illustrates a stereoscopic endoscope distal end portion and FIG. 2B illustrates a video image captured by the stereoscopic endoscope, the video image also containing a treatment tool. The endoscopes illustrated in FIGS. 1 and 2 include an objective lens 2, a treatment tool insertion hole 3, a treatment tool 4 and an emission port 8 (outlet of the emission unit, corresponding to a termination of the optical fiber). The endoscope distal end portion 1 is provided with at least the emission port 8 and objective lens 2.

Light (illumination light) for use to observe the inside of the subject is emitted through the emission port 8 to the inside of the subject. The illumination light may be guided from the light source 30 to the emission port 8 in the endoscope distal end portion 1 through the optical fiber 31 and lens as illustrated in FIG. 3 or an LED or the like may be installed near the emission port 8 at the endoscope distal end portion 1.

The objective lens 2 illustrated in FIGS. 1A to 2B is placed in the endoscope distal end portion 1 and receives the illuminating light released from the emission port 8 and reflected off the observed object and surrounding objects. The received light is focused on a light-receiving element such as a CCD installed in the endoscope 7 illustrated in FIG. 3, and an observation image is converted into an electric signal.

The control unit 18 illustrated in FIG. 3 is operated by an observer or observation assistant by being gripped outside the subject either manually or by a mechanical aid. Since the illuminating light is guided by an optical fiber in the endoscope of the type described in the present embodiment, a light introduction port 9 is provided in the control unit 18 and the optical fiber runs inside the endoscope 7 from the light introduction port 9 to the emission port 8. To carry a lot of light to the endoscope distal end portion, advisably the optical fiber is made up of about 10,000 small-diameter fibers bundled together.

The light source 30 contains a lamp light source. In particular, the present invention, according to which the light for in vivo observation and illumination for treatment tool observation differ in wavelength, may use independent lamp light sources for in vivo observation and treatment tool observation or adjust luminance on a wavelength-by-wavelength basis using a single lamp light source and color filters. When independent lamp light sources are used, a xenon lamp or halogen lamp is used as a light source for in vivo observation, and a xenon lamp which is relatively free of extreme wavelength irregularities in luminance from short wavelengths to long wavelengths is used suitably. Also, since only a specific wavelength is used according to application, the lamp light source is used in combination with color filters or an LED lamp is used. Also, as a light source for treatment tool observation, a wavelength which does not overlap with the light source for in vivo observation is chosen selectively, and thus a halogen lamp, xenon lamp, LED lamp, or a combination of any of these lamps with color filter is selected. In particular, since it is desirable that the wavelength for treatment tool observation does not overlap the wavelength for in vivo observation, blue color which is infrequently found in the body is suitable, and therefore a blue LED lamp or the like is used suitably. According to the present invention, the luminance of these lamp light sources can be adjusted independently. For that, applied voltages are set to be adjusted independently in some cases, and ND filters for adjustments are installed independently in other cases.

The lights emitted from the lamp light sources are combined when passing through the optical fiber and illuminate the inside of the body through the emission port 8. A LED light is sometimes installed in the endoscope distal end portion 1 rather than outside the endoscope as illustrated in FIG. 3. In that case, two LEDs—a LED for in vivo observation and a LED for treatment tool observation--may be installed inside the endoscope distal end portion 1 or only one of the LEDs may be installed in the endoscope distal end portion 1 with the other LED installed in the manner illustrated in FIG. 3.

The image captured by the endoscope distal end portion 1 is transmitted inside the endoscope and then transmitted to the video processor 20 through a signal cable 17.

The display 10 displays a video image to allow external observers to observe the video image captured by the endoscope. The endoscope is connected to the display 10 either through or without a video processor.

By using the endoscopic system configured as described above, the visibility of the treatment tool can be changed without affecting the in vivo observation image.

### EXAMPLE 1

Example 1 of the present invention will be described next. The endoscope used in the endoscopic system according to the present invention was a commercially available rigid endoscope 10 mm in diameter and 300 mm in length. A CCD with 1.23 million pixels was used.

In the present embodiment, a 300-watt xenon lamp was contained in the light source 30 for illumination for in vivo observation, and an emission spectrum of illuminating light for in vivo observation was formed by filtering with plural sharp cut filters (made by Sigma Koki Co., Ltd) in combination. To properly observe the red color, which is a main color of organs and the like, the emission spectrum of the illuminating light for in vivo observation which formed the second wavelength region was configured such that emission intensities equal to or higher than 50% a peak emission intensity would fall within a wavelength range of 620 nm to 670 nm. Regarding the illumination for treatment tool observation, which formed the first wavelength region, illuminating light for treatment tool observation was formed by applying a blue band pass filter made by Laser Create Corp. to a 100-watt white LED lamp. The emission spectrum was configured such that emission intensities equal to or higher than 50% a peak emission intensity would fall within a wavelength range of 435 nm to 475 nm. A spectral radiance spectrum was observed by illuminating a standard diffuser at an angle of 45 degrees and measuring luminance on a surface of the standard diffuser. The emission spectrum of each illuminating light after passage through the filter is illustrated in FIG. 4. In particular, in the state illustrated in FIG. 4, the treatment tool illuminating light has maximum spectral radiance. When the treatment tool was not illuminated, the treatment tool illuminating light was dimmed gradually from this brightness using a circular variable ND filter.

Fibers made of SiO₂ with an internal transmittance of 99% or more were used for the treatment tool. An ITO film was formed on the surface of the treatment tool by a sol-gel process.

The wavelength range for in vivo observation was 620 nm to 670 nm as with the illuminating light. Generally, the wavelength range of light for in vivo observation corresponds to the range of illuminating light for in vivo observation and overlaps a range of 400 to 710 nm, which is equal to or larger than a 1% range of a luminosity curve.

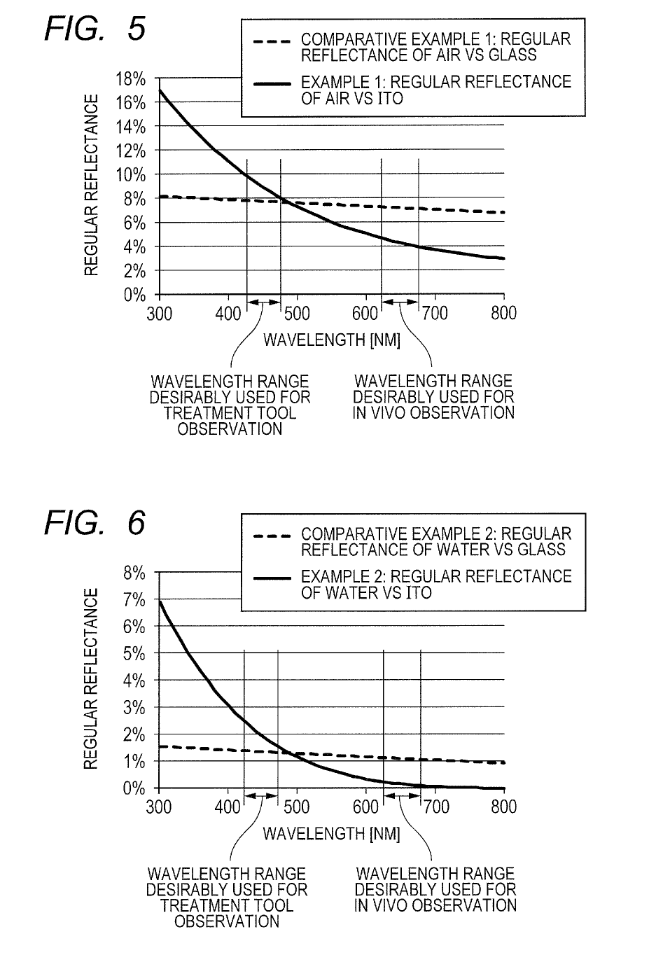

Also, the wavelength range for treatment tool observation was 435 to 475 nm. In air, the average value of the regular reflectance of an ITO film in the wavelength range for in vivo observation was 4.3%, and the average value of the regular reflectance of the treatment tool surface in the wavelength range for treatment tool observation was 9.1%, which was approximately twice as high as the regular reflectance for in vivo observation as illustrated in FIG. 5.

Using the endoscopic system constructed as described above and equipped with a treatment tool and illumination, the range of visibility changes of the treatment tool was checked. The changes in the visibility of the treatment tool were evaluated in terms of a ratio between changes in the average luminance of the treatment tool and surroundings thereof on the screen when only the illumination for in vivo observation was turned on and changes in the average luminance of the treatment tool and surroundings thereof on the screen when the illumination for treatment tool observation was turned on in addition to the illumination for in vivo observation.

This time, the numbers of gradations were compared using a monitor whose gradation-luminance characteristic was linear. On a 10-bit display, the number of gradations changed approximately 108% from 3.4 to 7.1 when the illumination for treatment tool observation was turned on. Furthermore, the 108% is accounted for by a special color originally not contained in the illumination for in vivo observation, which results in high visibility.

### COMPARATIVE EXAMPLE 1

A glass film whose major component was SiO₂ as with the base material of the optical fiber and whose reflectance had low wavelength dependence was formed on the surface of the treatment tool by the sol-gel process. Otherwise the treatment tool was created by the same method as Example 1. As a result, in air, the average value of the regular reflectance in the wavelength range for in vivo observation was 7.2% and the average value of the regular reflectance of the treatment tool surface in the wavelength range for treatment tool observation was 7.7%, which was approximately 1 times, and was much the same as, the regular reflectance for in vivo observation as illustrated in FIG. 5.

According to the above procedures, the range of visibility changes was from 5.5 gradations to 8.6 gradations, which was a change of only approximately 57%. That is, the visibility was not able to be doubled.

Also, Example 1 was able to achieve approximately twice the effect of Comparative Example 1.

### EXAMPLE 2

Example 2 differed from Example 1 in that evaluations were made in water by putting the endoscope distal end portion in water assuming the use of the endoscope distal end portion in a liquid such as amniotic fluid. In this case, the refractive index of the fiber approaches the refractive index of the fiber's surroundings, resulting in an extreme reduction of regular reflectance and thereby increasing transparency. The impact of changes in the degree of visibility becomes noticeable accordingly. As a result, the regular reflectance in the liquid at this time was as illustrated in FIG. 6: the average value of the regular reflectance in the wavelength range desirably used for in vivo observation was 0.18%, and the average value of the regular reflectance of the treatment tool surface in the wavelength range for treatment tool observation was 2.0%, which was ten times the corresponding value for in vivo observation. In the endoscopic system used in this operating environment, the number of gradations on a 10-bit display changed as much as approximately 497.3% from 0.16 to 0.98 when the illumination for treatment tool observation was turned on.

### COMPARATIVE EXAMPLE 2

Comparative Example 2 differed from Comparative Example 1 in that evaluations were made in water by putting the endoscope distal end portion in water assuming the use of the endoscope distal end portion in a liquid such as amniotic fluid. As a result, in air, the average value of the regular reflectance in the wavelength range for in vivo observation was 1.1% and the average value of the regular reflectance of the treatment tool surface in the wavelength range for treatment tool observation was 1.4%, which was 1.3 times, and was much the same as, the regular reflectance for in vivo observation as illustrated in FIG. 5.

The range of visibility changes in the endoscopic system evaluated in this way was from 12.54 gradations to 13.09 gradations, which was a change of only approximately 64%.

That is, Example 2 allowed visibility adjustment 8 times more than Comparative Example 2.

### EXAMPLE 3

Example 3 differed from Example 1 in that the wavelength of the illuminating light for in vivo observation was extended to a range of 450 nm to 710 nm. Compared to a comparative example, this improves visibility and reduces color discrepancy of in vivo observation images between the presence and absence of treatment tool observation light.

### EXAMPLE 4

Example 4 differed from Example 1 in that a film containing Zn₂SiO₄:Mn phosphor was formed on the treatment tool surface instead of an ITO coating, where the phosphor had an excitation wavelength of 250 to 400 nm and an emission color of green. Thus, the excitation wavelength was accommodated using illumination with a half-wavelength of 350 to 400 nm as the illumination for treatment tool observation. The range of visibility changes achieved in the endoscopic system evaluated in this way was from 5.48 gradations to 11.89 gradations, which was a change of approximately 117%.

The results of the examples and comparative examples are summarized in the table below.

TABLE 1
Amount of visibility adjustment (luminance ratio
of treatment tool portion with respect to unlit condition
of the treatment tool observation light)

| | |
|---|---|
| Example 1 | 108% |
| Comparative Example 1 | 57% |
| (conventional example) | |
| Example 2 | 497.30% |
| Comparative Example 2 | 64% |
| Example 4 | 117% |

The endoscopic system according to the present invention allows the visibility of the treatment tool to be changed, as required, depending on the wavelength of emitted light. As the visibility under the light in the wavelength region (a second wavelength region) for in vivo observation is reduced, the visibility of the treatment tool during in vivo observation becomes low while the visibility of the treatment tool under the light in the wavelength region (a first wavelength region) for treatment tool observation becomes high, and consequently the visibility during treatment tool observation becomes high in a relative sense. Furthermore, by adjusting the intensity of the illuminating light for treatment tool observation, the endoscopic system according to the present invention allows the visibility of the treatment tool to be adjusted.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-056034, filed Mar. 19, 2013, which is hereby incorporated by reference herein in its entirety.

What is claimed is:

1. An endoscopic system comprising:
an emission unit; and
a treatment tool,
wherein the emission unit emits light in a first wavelength region and light in a second wavelength region, and
visibility of the treatment tool under the light in the first wavelength region is higher than visibility of the treatment tool under the light in the second wavelength region.

2. The endoscopic system according to claim 1, wherein:
the treatment tool includes a treatment tool body and a coating layer; and
the coating layer is made of a material different in refractive index from the treatment tool body.

3. The endoscopic system according to claim 2, wherein:
the treatment tool body is made of SiO₂; and
the coating layer is made of ITO.

4. The endoscopic system according to claim 1, wherein the second wavelength region is from 450 nm to 700 nm, both inclusive.

5. The endoscopic system according to claim 1, wherein the treatment tool further includes a phosphor configured to be excited to emit light at a wavelength of 450 nm or below.

6. The endoscopic system according to claim 1, wherein the phosphor contains Zn₂SiO₄.

7. The endoscopic system according to claim 1, wherein the emission unit includes a first emission unit adapted to emit the light in the first wavelength region and a second emission unit adapted to emit the light in the second wavelength region.

8. The endoscopic system according to claim 1, wherein the emission unit includes a light source and an optical fiber.

9. The endoscopic system according to claim 1, wherein at least one of the first emission unit and the second emission unit has a light-emitting element.

10. The endoscopic system according to claim 1, comprising a treatment tool insertion hole used to insert the treatment tool.

11. The endoscopic system according to claim 1, wherein the emission unit includes a filter adapted to selectively pass light of a specific wavelength.

12. The endoscopic system according to claim 1, further comprising:
a plurality of the imaging units; and
a circuit adapted to generate a three-dimensional image based on information obtained by the plurality of imaging units.

13. An imaging method using an endoscope equipped with an emission unit, an imaging unit, and a treatment tool,
wherein the emission unit operates in a first mode in which light in a first wavelength region is emitted and a second mode in which light in a second wavelength region is emitted,
the imaging method comprising a first observation step of observing the treatment tool using illumination in the first mode, and
a second observation step of observing an object under observation using illumination in the second mode.
